(19) United States
(12) Patent Application Publication
(10) Pub. No.: US 2018/0237861 A1
(43) Pub. Date: Aug. 23, 2018

(54) PERSONALIZED TREATMENT OF DISEASES AND DISORDERS

(71) Applicant: CHAMPIONS ONCOLOGY, INC., Hackensack, NJ (US)

(72) Inventors: David SIDRANSKY, Pikesville, MD (US); Keren PAZ, Tenafly, NJ (US)

(21) Appl. No.: 15/752,296

(22) PCT Filed: Aug. 10, 2016
(86) PCT No.: PCT/US16/46330

§ 371 Date: Feb. 13, 2018

Related U.S. Application Data
(60) Provisional application No. 62/204,536, filed on Aug. 13, 2015.

(51) Int. Cl. C12Q 1/6886; A61K 49/00; A61K 39/00; A61P 35/00; A01K 67/027

(52) U.S. Cl. CPC C12Q 1/6886; A61K 49/0008; A61K 39/0011; A61P 35/00; A01K 2267/0331; C12Q 2600/106; A01K 2207/12; A01K 2227/105; A01K 67/0278

(57) ABSTRACT

The invention relates generally to a personalized treatment of a disease or disorder using a humanized non-human mammal model. Specifically, the invention relates to a use of a humanized non-human mammal model for identifying effective therapeutic molecules to provide a personalized treatment of a disease or disorder.

### CROSS-REFERENCE TO RELATED APPLICATIONS

This application claims priority to and the benefit of U.S. Provisional Patent Application 62/204,536, filed Aug. 13, 2015, which is incorporated by reference herein in its entirety.

### FIELD OF THE INVENTION

The invention relates generally to a personalized treatment of a disease or disorder using a humanized non-human mammal model. Specifically, the invention relates to a use of a humanized non-human mammal model for identifying effective therapeutic molecules to provide a personalized treatment of a disease or disorder.

### BACKGROUND OF THE INVENTION

Before personalized medicine, most patients with a specific type and stage of cancer received the same treatment. However, it has become clear to doctors that some treatments worked well for some patients and not as well for others. Thus, there is a need to develop an effective, personalized therapeutic molecule or vaccine effective for a particular patient. Personalized treatment strategies may be more effective and cause fewer side effects than would be expected with standard treatments.

Tumors develop due to mutations in a person's DNA, causing production of mutated proteins comprising neo-epitopes not present within the corresponding normal protein produced by the host. Many of these neo-epitopes stimulate T-cell response and result in the destruction of early-stage cancerous cells by the immune system. In cases of established cancer, however, the immune response is insufficient. In other instances, development of effective, long term vaccines that target tumor antigens in cancer, but not specifically targeting the neo-epitopes thereof, have proven difficult. A major reason for this is that T cells specific for tumor self-antigens are eliminated or inactivated through mechanisms of tolerance.

Neo-epitopes are epitopes present within a protein associated with a disease, for example cancer, wherein the specific “neo-epitope” is not present within the corresponding normal protein associated with a non-diseased subject or tissue therein. Neo-epitopes may be challenging to identify because they are rare and can vary from person to person. Additionally, identifying those neo-epitopes that differ enough from the host to elicit an actual immune response presents yet another challenge.

The use of neo-epitopes is a promising therapeutic avenue for immunotherapy of cancer and other diseases, with durable objective responses observed in patients. However, current animal models often fail to pinpoint immunotherapies with the greatest clinical potential due in part to differences between human and murine immune systems.

Accordingly, there exists a need for reliable animal models to test neo-epitopes in the context of a human immune system.

### SUMMARY OF THE INVENTION

Methods of the invention are useful for providing a therapeutic that activates an individual's immune system against specific disease-related cells. The therapeutic is provided by analyzing sequences to identify epitopes specific to the diseased cells. The identified disease-specific epitopes can be synthesized and tested in an animal that has been engineered to include components of the human immune system. One or more of the epitopes that exhibit good results in the animal are thus provided as effective epitopes for activating an individual's immune system against the diseased cells. A therapeutic can be created that includes one or more of those effective epitopes and the therapeutic can be used for treatment.

Sequence analysis to identify disease-specific epitopes may include sequencing nucleic acid from both diseased and healthy cells obtained from a person to identify a set of mutations that characterize the diseased cells and generating polypeptide sequences encoded by the nucleic acid that include those mutations. The polypeptide sequence are inputed into a tool, such as an artificial neural network (ANN) that has been trained on peptide:MHC affinity measurements (e.g., the NetMHC server), that identifies, and predicts the MHC binding affinity of, the disease-specific epitopes in the polypeptide sequences.

Testing the disease-specific epitopes may include administering the epitopes to a humanized animal, i.e., an animal with at least parts of a human immune system. The animal may also include a human tumor xenograft. The effectiveness of the epitopes may be established by observing their effects on the animal, for example, by observing inhibition of tumor growth.

Since the epitopes are selected from sequences that include mutations characterizing the diseased cells, the therapeutic will target diseased cells preferentially over healthy cells. Since the mutations are determined by analyzing sequences from the person, the epitopes may include those neo-epitopes, very rare epitopes, or those that vary from person to person—i.e., those epitopes that could not be provided by other, non-personalized methods. Since the MHC binding affinity is predicted for each epitope using a tool such as a trained neural network, e.g., as embodied in the NetMHC server, a very large number of epitopes may be initially identified to ensure that all potentially valuable and effective epitopes are swept in, and then that very large set can be winnowed to identify the most potentially effective disease-specific epitopes. Since the epitopes are tested in animals that exhibit human immune components, not only is the actual efficacy of those epitopes screened but potential adverse effects in humans may also be identified and ruled out. Thus, methods of the invention can be used to rapidly identify and screen personalized epitopes to provide effective disease-specific epitopes that may be used in treatment of the person.

In one aspect, the invention provides a method for identifying an effective therapeutic molecule to treat a disease in a subject, the method comprising: obtaining a biological sample associated with said disease from said subject; determining a genetic alteration associated with said disease in said sample; identifying one or more disease-specific epitopes, and thereby identifying one or more antigenic epitope peptides each having said one or more disease-specific epitopes; providing a humanized non-human mammal (e.g. mouse), wherein said non-human mammal is an immune-compromised non-human mammal reconstituted with a human immune system; testing the therapeutic effect of said one or more antigenic epitope peptides for treating said disease in said subject by administering said one or more antigenic epitope peptides to said humanized non-human mammal; evaluating the therapeutic effect of said one or more antigenic epitope peptides; and identifying an antigenic epitope peptide effective to treat said disease in said subject, thereby identifying said effective therapeutic molecule to treat said disease in said subject. In an exemplary embodiment, the non-human mammal is a mouse.

In some embodiments, the method comprises the steps of sequencing at least a portion of the a patient's RNA or DNA obtained from both a healthy tissue and a diseased tissue, to produce a healthy tissue RNA or DNA sequence and a diseased tissue RNA or DNA sequence; comparing the healthy tissue RNA or DNA sequence and the diseased tissue RNA or DNA sequence; and identifying differences between the healthy tissue RNA or DNA sequence and the diseased tissue RNA or DNA sequence to produce a variant DNA marker set; and identifying disease-specific epitopes using a predictive algorithm. In one embodiment, the coding region of the patient's whole genome is evaluated for sequence mutations (e.g., single base mutation, insertion, and deletion). In another embodiment, the patient's transcriptomes are evaluated.

In one embodiment, the disease is a cancer disease. In some embodiments, the therapeutic effect of said one or more antigenic epitope peptides can be evaluated or tested in a humanized mouse comprising a human tumor xenograft, wherein said xenograft is associated with said disease.

In another aspect, the invention provides a method for treating a disease (e.g., cancer disease) in a patient, the method comprising: obtaining a biological sample associated with said disease from said subject; determining a genetic alteration associated with said disease in said sample; identifying one or more disease-specific epitopes, and thereby identifying one or more antigenic epitope peptides each having said one or more disease-specific epitopes; providing a humanized non-human mammal (e.g., mouse), said non-human mammal is an immune-compromised non-human mammal reconstituted with a human immune system; testing the therapeutic effect of said one or more antigenic epitope peptides in treating said disease in said subject by administering said one or more antigenic epitope peptides to said humanized non-human mammal; evaluating the therapeutic effect of said one or more antigenic epitope peptides; and identifying an antigenic epitope peptide effective to treat said disease in said subject; and administering said effective therapeutic agent to said patient, thereby treating said disease in said patient.

In a further aspect, the invention provides a method for providing a personalized treatment to treat a tumor in a patient.

Aspect of the invention includes a method for providing a therapeutic for a person with a disease. The method includes obtaining a nucleic acid sequence from one or more disease-affected cells from the person and identifying—using computer system comprising at least one processor coupled to a memory subsystem—a plurality of epitopes, wherein each epitope is encoded by a portion of the sequence that differs from a corresponding sequence from healthy cells from the person by at least one variant. The computer system is used to select at least one of the plurality of epitopes based on a predicted MHC binding affinity of that epitope. The method further includes observing a therapeutic effect of the selected epitope on a non-human animal that has been engineered to include parts of a human immune system and identifying the selected epitope as a therapeutic to treat the disease in the person. Obtaining the nucleic acid sequence may include sequencing nucleic acid from the one or more disease-affected cells and optionally sequencing additional nucleic acid from the healthy cells to obtain a healthy-type sequence. Identifying the plurality of epitopes may include comparing the sequence to the healthy-type sequence to identify the at least one variant.

In some embodiments, identifying the plurality of epitopes includes translating the nucleic acid sequence into amino acid sequence, excluding portions of the sequence that wholly match the corresponding sequence from the healthy cells, and storing the amino acid sequences in a tangible memory device within the memory system. In certain embodiments, selecting the at least one of the plurality of epitopes based on a predicted MHC binding affinity of that epitope includes providing the plurality of epitopes as an input to a program that predicts affinities using an artificial neural network (ANN) trained on peptide:MHC affinity measurement data.

The non-human animal may be an immune-compromised mouse reconstituted with a human immune system. In some embodiments, the non-human animal is an immune-deficient nude mouse reconstituted with a human immune system. The mouse may be a Non-Obese Diabetic (NOD) Shi-Scid IL-2R γnull (NOG) mouse. In certain embodiments, the non-human animal is a mouse is reconstituted with human CD34+ cells (preferably, after a pre-determined time of reconstitution, the mouse is capable of providing mature human CD45+ cells). The mouse may include hCD3+, hCD4+, and hCD8+ cells.

In certain embodiments, the disease is cancer. The non-human animal may be a mouse with an xenograft of a human tumor (e.g., subcutaneously implanted in the mouse). The human tumor xenograft may be a melanoma tumor graft, a colorectal tumor graft, a breast tumor graft, a lung tumor graft, a xenograft of a human mesenchymal chrondrosarcoma, a xenograft of a human leiomyosarcoma, or a xenograft of a human non-small cell lung cancer. Preferably, the mouse exhibits phenotypic stability of the tumor.

Other features and advantages of the present invention will become apparent from the following detailed description examples and FIGURES. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 illustrates a flowchart of a method for identifying an effective therapeutic molecule to treat a disease in a subject, according to one embodiment of the invention.

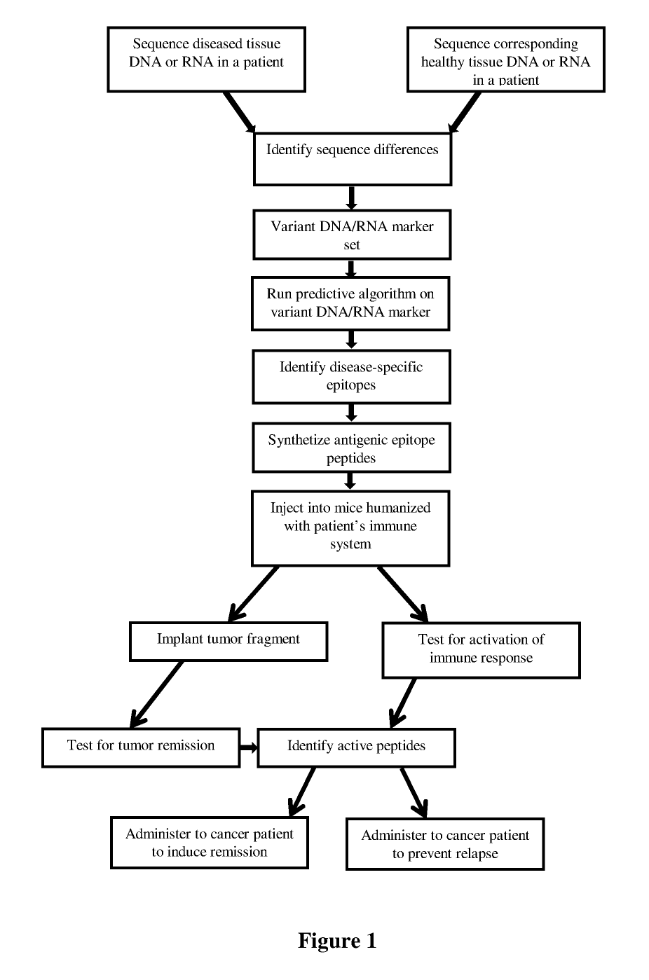

### DETAILED DESCRIPTION OF THE INVENTION

The invention provides a personalized treatment of a disease or disorder using a humanized non-human mammal model. Specifically, the invention provides a use of a humanized non-human mammal model for identifying effective therapeutic molecules to provide a personalized treatment of a disease or disorder.

The inventors of the instant application have developed systems and methods that combine two innovative approaches. Specifically, the present invention combines the humanized mouse model approach with the personalized genome analyses so as to identify an effective therapeutic antigen for treating a disease (e.g., cancer).

In one aspect, provided herein is a method for identifying an effective therapeutic molecule to treat a disease in a subject, the method comprising: obtaining a biological sample associated with said disease from said subject; determining a genetic alteration associated with said disease in said sample; identifying one or more disease-specific epitopes, and thereby identifying one or more antigenic epitope peptides each having said one or more disease-specific epitopes; providing a humanized non-human mammal (e.g. mouse), wherein said non-human mammal is an immune-compromised non-human mammal reconstituted with a human immune system; testing the therapeutic effect of said one or more antigenic epitope peptides for treating said disease in said subject by administering said one or more antigenic epitope peptides to said humanized non-human mammal; evaluating the therapeutic effect of said one or more antigenic epitope peptides; and identifying an antigenic epitope peptide effective to treat said disease in said subject, thereby identifying said effective therapeutic molecule to treat said disease in said subject. In an exemplary embodiment, the non-human mammal is a mouse.

The terms “biological sample,” as used herein, may refer to any sample prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In a particular embodiment, the biological sample is a tumor tissue.

The terms “genetic alteration,” as used herein, may refer to any type of genetic alteration, for example, but not limited to, a mutation, a translocation, and a copy number variation.

In an exemplary embodiment, genetic alterations associated with the disease can be determined by any suitable methods known to one of skilled in the art. In one example, the CancerXome™ approach developed by Personal Genome Diagnostics, Inc. is used to determine genetic alterations.

In one embodiment, nucleic acids (e.g., RNA or DNA) are isolated from diseased and healthy biological samples by techniques well-known in the art. In some embodiments, all or a portion of a patient's genome is isolated and sequenced by sequencing methods well-known in the art. High-throughput DNA sequencing methods are known in the art and include, for example, the HiSeq™2000 system by Illumina® Sequencing Technology, which uses a large parallel sequencing-by-synthesis approach to generate billions of bases of high-quality DNA sequence per run.

In certain embodiments, particular portions of the patient's genome are sequenced, depending on the disease. In a preferred embodiment, the entire genome or transcriptome is sequenced. In one embodiment, the coding region of the patient's whole genome is evaluated for sequence mutations (e.g., single base mutation, insertion, and deletion). In another embodiment, the patient's transcriptomes are evaluated. The genome may be sequenced to a shallow depth or a deep depth, allowing coverage of less or more portions of the genome or transcriptome. In some embodiments, further in-depth computational analyses using CHASM, Digital Karyotyping, and other approaches can be performed. Such approaches may allow for the differentiation of passenger (unimportant mutations) from, for example, oncogenic mutations.

In one example, one or more portions of a patient's RNA or DNA obtained from both a healthy tissue and a diseased tissue are sequenced to produce a healthy tissue RNA or DNA sequence and a diseased tissue RNA or DNA sequence. The healthy tissue RNA or DNA sequence are compared with the diseased tissue RNA or DNA sequence. The differences between the healthy tissue RNA or DNA sequence and the diseased tissue RNA or DNA sequence are identified to produce a variant DNA marker set.

In a specific embodiment, analyzing the variant DNA or RNA marker set to identify a disease-specific epitope set comprises using a predictive algorithm that predicts the ability of epitope peptides to bind MHC molecules. For example, in certain embodiments, polypeptide sequences are generated that are encoded by the nucleic acid that includes the variant marker set. The polypeptide sequence can be used as inputs to a tool such as an artificial neural network (ANN) like the one provided by the NetMHC server, e.g., as described in Lundegaard et al., NetMHC-3.0: accurate web accessible predictions of human, mouse, and monkey MHC class I affinities for peptides of length 8-11, Nucleic Acids Research 36:w509-w512, which is incorporated by reference herein. The NetMHC server provides an ANN that is trained on a large number of published quantitative peptide:MHC affinity measurements, eluted peptide data from the SYFPEITHI database, and proprietary affinity data. Where methods of the invention are implemented using a system of the invention, the system may include a computer comprising a processor coupled to a tangible memory device. The computer may send sequences to the NetMHC server as, for example, a list of peptides with a defined length (e.g., 8-11 residues) or all possible sub-peptides hosted within those polypeptides that include the variant marker set. The computer can be used to upload the input in the FASTA format, or as peptides all of equal length with one peptide per line. The server will accept a maximum of 5000 sequences per submission; each sequence not more than 20 000 amino acids with a minimum length corresponding to the selected length of prediction (see subsequently). The NetMHC server predicts binding affinity of the peptides and provides raw text with a columns for the epitope sequence and predicted affinity, among others. The computer can be used to receive the predicted affinities of the disease specific epitope set from the NetMHC server or from another computer or server that implements a predictive algorithm for MHC binding. That approach takes advantage of the neural network algorithm implemented by the NetMHC server. Other algorithms may be used. For example, a neural network may be implemented locally (e.g., using a computer system) and trained on published affinity data and then fed the sequences from the person.

Optionally, the disease-specific epitope set is refined to provide an MHC-restricted disease-specific epitope set. For example, MHC I-restricted epitopes of the K, D or L alleles can be provided. MHC-restricted epitope sets can be produced by determining binding of a peptide containing the epitope to an MHC-allele-specific peptide.

Predictive algorithms are known in the art and fully described, for example, in PCT Patent Application Publication WO 2014/052707, U.S. Patent Application Publications US 20040096892 and US 20130210645, all of which are incorporated by reference herein in their entirety.

Specifically, the DNA (or RNA) sequence differences between the healthy and diseased biological samples are analyzed by an epitope predictive algorithm. In one embodiment, such algorithm produces a list of potential disease-specific epitopes for an individual patient, and gives each epitope a numerical score. In the current state of the art, a high score implies a good probability of the epitope being able to immunize, and a low (including a negative) score implies a poor probability of the epitope being able to immunize.

In some embodiments, methods of the invention include steps implemented using a computer system of the invention that includes at least one processor coupled to a memory system and one or more input/output devices. The computer system may include any suitable components such as one or more desktop, laptop, or server computer. Each computer preferably includes at least one processor coupled to a memory system and one or more input/output devices. A memory subsystem preferably includes one or more memory devices operationally linked, such as a RAM chip and hard drive connected via a motherboard. Preferably, a memory subsystem includes at least one tangible, non-transitory memory device such as a hard drive, solid state drive, optical disk, or other such computer readable medium. A computer system of the invention may be used to obtain a nucleic acid sequence from one or more disease-affected cells from a person and identifying a plurality of epitopes using the methods described herein. Preferably each epitope is encoded by a portion of the sequence that differs from a corresponding sequence from healthy cells from the person by at least one variant. The system may be used to select at least one of the plurality of epitopes based on a predicted MHC binding affinity of that epitope, e.g., by using an ANN or other algorithm suitable for identifying epitopes.

In one aspect, one of skilled in the art can modify the one or more antigenic epitope peptides to maximize antigenicity. In one example, the one or more antigenic epitope peptides can be placed in a suitable vector or carrier, known to one of skilled in the art.

The therapeutic effect of one or more antigenic epitope peptides can be tested by administering the one or more antigenic epitope peptides to humanized non-human mammal model. The term “humanized”, as used herein refers to an immunodeficient mammal that harbors a population of heterogeneous immune cells that were introduced into it. The source of the heterogeneous immune cells may be either a donor mammal, or another humanized mammal.

Examples of non-human mammals include, but are not limited to, laboratory animals (e.g., rats, mice, hamsters, guinea pigs, monkeys, and apes), farm animals (e.g., cows, pigs, and horses), domesticated animals (e.g., dogs, cats, rabbits, and horses), human companion animals, zoo animals, and wild animals.

In a particular embodiment, the non-human mammal model is a mouse model. The mouse model of the invention may be an immune-compromised or an immune-deficient nude mouse. Any suitable mouse can be used to develop a mouse model of the invention. In an exemplary embodiment, the mouse is a Non-Obese Diabetic (NOD) Shi-Scid IL-2R γ^{null} (NOG) mouse. Other examples of mouse include, but are not limited to, Scid mouse, NOD/Shi mouse, IL-2R γ^{null} mouse, NOD/Sci-Scid mouse.

The non-human mammal, for example, mouse can be humanized by any suitable method known to one of skilled in the art. Methods for humanizing a mouse are well known in the art and fully described in U.S. Pat. No. 8,604,271; U.S. Pat. No. 8,071,839; U.S. Pat. No. 6,676,924; U.S. Pat. No. 5,874,540; U.S. Pat. No. 8,658,154; U.S. Pat. No. 8,110,720; and U.S. Pat. No. 5,777,194 as well as U.S. Patent Application Publications US 2013/0291134; US 2013/0217043; US 2012/0066780; US 2005/0089538; and US 2002/0018750, all of which are incorporated by reference herein in their entirety.

In some embodiments, the non-human mammal (e.g., mouse) model is reconstituted with human CD34+ cells. In one embodiment, after a pre-determined time of reconstitution (e.g., 4-10 weeks post hCD34+ reconstitution), the reconstituted non-human mammal (e.g., mouse) is capable of providing mature human CD45+ cells. In a particular embodiment, the reconstituted non-human mammal (e.g., mouse) is capable of providing hCD3+, hCD4+, and hCD8+ cells.

In some embodiments, the humanized non-human mammal (e.g., mouse) model of the invention can be developed by adoptive transfer of splenocytes. This approach is fully described in U.S. Patent Application 62/165,464, which is incorporated by reference herein in its entirety.

In one example, in the humanized mouse model of the invention, one or more human tumor xenografts can implanted, for example, subcutaneously implanted by any suitable method known in the art. Methods for implanting a tumor xenograft in a mouse are well known in the art and fully described in PCT patent application publications WO 2008/143795 and WO 2008/140751, which are incorporated by reference herein in their entirety.

Depending on a disease treatment, a suitable tumor graft can be used. In one example, the human tumor xenograft is a melanoma tumor graft. In another example, the human tumor xenograft is a colorectal tumor graft. In another example, the human tumor xenograft is a breast tumor graft. In another example, the human tumor xenograft is a lung tumor graft. In another example, the human tumor xenograft is a xenograft of a human mesenchymal chrondrosarcoma. In another example, the human tumor xenograft is a xenograft of a human leiomyosarcoma. In another example, the human tumor xenograft is a xenograft of a human non-small cell lung cancer.

After implantation of tumor grafts, phenotypic stability of the tumor can be evaluated. In one embodiment, after implantation of tumor grafts, engraftment and growth rates can be evaluated. In a particular embodiment, the mouse model of the invention exhibits phenotypic stability of the tumor.

In another aspect, provided herein is a method for identifying an effective therapeutic regimen for treating a tumor in a patient. The method may include the steps of providing a humanized mouse having a human tumor xenograft of the invention; testing one or more disease associated antigens of the invention to evaluate their effect on tumor growth inhibition in said mouse; and identifying an effective therapeutic antigen to treat said patient.

In addition to the antigens of the invention, the therapeutic regimen may include any suitable type of therapeutic treatments that need to be evaluated on tumor growth. In a particular embodiment, the therapeutic regimen is a therapeutic agent. Examples of a therapeutic agent include, but not limited to, small molecule compounds and large molecules (e.g., antibodies). In a particular embodiment, the therapeutic agents are molecules targeting immune checkpoints.

In another aspect, provided herein is a method for treating a tumor in a subject (e.g., human patient), the method comprising: providing a humanized mouse comprising a human tumor xenograft; testing one or more therapeutic antigens of the invention to evaluate their effect on tumor growth inhibition in said mouse; identifying an effective therapeutic antigen; and treating said tumor in said subject.

In one example, the identified effective therapeutic antigen can be used as a vaccine.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented. In one example, the terms “treat” and “treatment” refer to inhibiting tumor growth.

The non-human mammal (e.g., mouse) model of the invention can be used to test and identify one or more effective therapeutic molecules (e.g., antigens) in order to treat any disease or disorder (e.g., cancer/tumor) in a subject. Examples of cancers/tumors which may be treated include, but not limited to, a melanoma, a colorectal cancer, a breast cancer (including HER2+ and metastatic), a lung cancer. Additional examples of cancers/tumors which may be treated include, but not limited to, a bladder cancer, a prostate cancer, an ovarian cancer, and a gastrointestinal cancer. Examples of a lung cancer include, but are not limited to a small cell lung cancer (SCLC) or a non-small cell lung cancer (NSCLC).

Cancers to be treated may include primary tumors and secondary or metastatic tumors (including those metastasized from lung, breast, or prostate), as well as recurrent or refractory tumors. Recurrent tumors encompass tumors that appear to be inhibited by treatment, but recur up to five years, sometimes up to ten years or longer after treatment is discontinued. Refractory tumors are tumors that have failed to respond or are resistant to treatment with one or more conventional therapies for the particular tumor type. Refractory tumors include those that are hormone-refractory (e.g., androgen-independent prostate cancer; or hormone-refractory breast cancer, such as breast cancer that is refractory to tamoxifen); those that are refractory to treatment with one or more chemotherapeutic agents; those that are refractory to radiation; and those that are refractory to combinations of chemotherapy and radiation, chemotherapy and hormone therapy, or hormone therapy and radiation.

Therapy may be “first-line”, i.e., as an initial treatment in patients who have had no prior anti-cancer treatments, either alone or in combination with other treatments; or “second-line”, as a treatment in patients who have had one prior anti-cancer treatment regimen, either alone or in combination with other treatments; or as “third-line,” “fourth-line,” etc. treatments, either alone or in combination with other treatments.

Therapy may also be given to patients who have had previous treatments which have been partially successful but are intolerant to the particular treatment. Therapy may also be given as an adjuvant treatment, i.e., to prevent reoccurrence of cancer in patients with no currently detectable disease or after surgical removal of tumor.

The mouse model of the invention can also be used for providing a personalized treatment to treat a tumor in a patient. Accordingly, in another aspect, provided herein is a method for providing a personalized treatment to treat a tumor in a patient, the method comprising: providing a humanized mouse comprising a tumor xenograft obtained from said patient; testing one or more therapeutic agents to evaluate the effect of said agents on tumor growth inhibition in said mouse; identifying an effective therapeutic agent; and treating said tumor in said patient, thereby providing a personalized treatment to treat said tumor in said patient.

Any patent, patent application publication, or scientific publication, cited herein, is incorporated by reference herein in its entirety.

The following examples are presented in order to more fully illustrate the preferred embodiments of the invention. They should in no way be construed, however, as limiting the broad scope of the invention.

### EXAMPLES

### Example 1

### A Humanized Mouse Model for Preclinical Testing of Therapeutic Antigens

The diseased tissue (e.g. cancer tissue) can be harvested from a patient together with the corresponding healthy tissue. DNA or RNA can be extracted from both samples using standard methods. The extracted DNA or RNA can be subjected to high throughput sequencing analysis to obtain genome or transcriptome sequence of a patient's healthy and cancer cells. The two sequences are then analyzed to identify the differences, such as mutations, translocations, deletions or amplifications. The identified differences can be further analyzed using the CancerXome™ approach developed by Personal Genome Diagnostics, Inc. to distinguish true cancer-associated changes from false positives and artifacts. The identified pool of cancer-associated changes in genome or transcriptome can be additionally analyzed to identify the sequences that are likely to be most effective as cancer-specific antigens. Thus, the peptide sequences corresponding to candidate mutation can be analyzed for their ability to interact with immunoactivating factors, such as MHC I using predictive sequence analysis algorithms. Such analysis will generate a numerical score for each candidate peptide sequence, allowing for selection of the candidate sequences with the greatest immunogenic potential.

The selected peptides can be synthesized and their ability to activate immune response can be tested in humanized mice that were reconstituted with the patient's immune system. The ability of the selected peptides to affect the cancer can also be tested in humanized mice that were xenografted with the same cancer tissue that was used to identify the cancer-associated changes in genome or transcriptome. The peptides that displayed either the ability to activate immune system or to affect cancer can be subsequently administered directly to the patient in order to treat this cancer, or to vaccinate the patient in order to prevent relapse.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method for identifying an effective therapeutic molecule to treat a disease in a subject, the method comprising:
obtaining a biological sample associated with said disease from a subject;
determining one or more genetic alterations associated with said disease in said sample;
identifying one or more disease-specific epitopes, and thereby identifying one or more antigenic epitope peptides each having said one or more disease-specific epitopes;
providing a humanized non-human mammal, said non-human mammal is an immune-compromised non-human mammal reconstituted with a human immune system;
testing the therapeutic effect of said one or more antigenic epitope peptides in treating said disease in said subject by
administering said one or more antigenic epitope peptides to said humanized non-human mammal;
evaluating the therapeutic effect of said one or more antigenic epitope peptides; and
identifying an antigenic epitope peptide effective to treat said disease in said subject,
thereby identifying said effective therapeutic molecule to treat said disease in said subject.

2. The method of claim 1, wherein the genetic alteration is determined by sequencing at least a portion of RNA or DNA obtained from a healthy tissue and a diseased tissue, to produce a healthy tissue RNA or DNA sequence and a diseased tissue RNA or DNA sequence; comparing the healthy tissue RNA or DNA sequence and the diseased tissue RNA or DNA sequence; and identifying differences between the healthy tissue RNA or DNA sequence and the diseased tissue RNA or DNA sequence to produce a variant DNA marker set.

3. The method of claim 1, wherein the said disease-specific epitopes correspond to said disease-specific genetic alterations.

4. The method of claim 2, wherein sequencing is transcriptome sequencing.

5. The method of claim 2, wherein DNA or RNA sequencing is high-throughput sequencing.

6. The method of claim 1, wherein identifying disease-specific epitopes comprises using a predictive algorithm.

7. The method of claim 1, wherein said disease is cancer.

8. The method of claim 1, wherein said non-human mammal is a mouse, wherein said mouse is an immune-compromised mouse reconstituted with a human immune system.

9. The method of claim 1, wherein said non-human mammal is a mouse, wherein said mouse is an immune-deficient nude mouse reconstituted with a human immune system.

10. The method of claim 9, wherein said mouse is a Non-Obese Diabetic (NOD) Shi-Scid IL-2R γ^{null} (NOG) mouse.

11. The method of claim 1, wherein said non-human mammal is a mouse, wherein said mouse is reconstituted with human CD34+ cells.

12. The method of claim 11, wherein, after a pre-determined time of reconstitution, said mouse is capable of providing mature human CD45+ cells.

13. The method of claim 12, wherein said mouse comprises hCD3+, hCD4+, and hCD8+ cells.

14. The method of claim 7, wherein said humanized non-human mammal is a mouse, wherein said mouse further comprises an xenograft of a patient's tumor.

15. The method of claim 14, wherein said human tumor xenograft is subcutaneously implanted in said mouse.

16. The method of claim 14, wherein said human tumor xenograft is a melanoma tumor graft, a colorectal tumor graft, a breast tumor graft, a lung tumor graft, a xenograft of a human mesenchymal chrondrosarcoma, a xenograft of a human leiomyosarcoma, or a xenograft of a human non-small cell lung cancer.

17. The method of claim 14, wherein said mouse exhibits phenotypic stability of said tumor.

18. A method for treating a disease in a subject, the method comprising:
obtaining a biological sample associated with said disease from said subject;
determining a genetic alteration associated with said disease in said sample;
identifying one or more disease-specific epitopes, and thereby identifying one or more antigenic epitope peptides each having said one or more disease-specific epitopes;
providing a humanized non-human mammal, said non-human mammal is an immune-compromised non-human mammal reconstituted with a human immune system;
testing the therapeutic effect of said one or more antigenic epitope peptides in treating said disease in said subject by administering said one or more antigenic epitope peptides to said humanized non-human mammal;
evaluating the therapeutic effect of said one or more antigenic epitope peptides; and
identifying an antigenic epitope peptide effective to treat said disease in said subject; and administering said effective therapeutic agent to said patient, thereby treating said disease in said patient.

19. A method for providing a personalized treatment to treat a tumor in a subject, the method comprising:
obtaining a biological sample associated with said disease from said subject;
determining a genetic alteration associated with said disease in said sample;
identifying one or more disease-specific epitopes, and thereby identifying one or more antigenic epitope peptides each having said one or more disease-specific epitopes;
providing a humanized mouse, said mouse comprising immune cells of said subject;
testing the therapeutic effect of said one or more antigenic epitope peptides in treating said disease in said subject by administering said one or more antigenic epitope peptides to said humanized mouse;
evaluating the therapeutic effect of said one or more antigenic epitope peptides; and identifying an antigenic epitope peptide effective to treat said disease in said subject; and administering said effective therapeutic agent to said patient, thereby treating said disease in said patient.

20. A method for identifying an effective therapeutic molecule to treat a cancer in a subject, the method comprising:
obtaining a healthy tissue sample and said subject's cancer tissue sample;
isolating RNA or DNA from said healthy tissue sample and cancer tissue sample; sequencing at least a portion of said RNA or DNA obtained from both said healthy tissue sample and cancer tissue sample to produce a healthy tissue RNA or DNA sequence and a cancer tissue RNA or DNA sequence;
comparing the healthy tissue RNA or DNA sequence and the cancer tissue RNA or DNA sequence and identifying differences between the healthy tissue RNA or DNA sequence and the diseased tissue RNA or DNA sequence to produce a variant marker set;
analyzing the variant marker set to produce a tumor-specific epitope set, wherein the tumor-specific epitope set comprises one or more tumor-specific epitopes;
providing a numerical score for each epitope in the tumor-specific epitope set;
identifying one or more tumor-specific antigenic epitope peptides, or one or more antigenic peptides each having one or more the tumor-specific epitopes;
providing a humanized mouse, wherein said mouse is an immune-compromised mouse reconstituted with a human immune system;
testing said one or more antigenic peptides to evaluate the effect of said tumor-specific epitopes on activation of human immune system in said mouse; and
identifying an effective tumor-specific antigenic peptides to treat said cancer in said patient.

21. A method for providing therapeutic for a person with a disease, the method comprising:
obtaining a nucleic acid sequence from one or more disease-affected cells from the person;
identifying—using computer system comprising at least one processor coupled to a memory subsystem—a plurality of epitopes, wherein each epitope is encoded by a portion of the sequence that differs from a corresponding sequence from healthy cells from the person by at least one variant;
selecting at least one of the plurality of epitopes based on a predicted MHC binding affinity of that epitope;
observing a therapeutic effect of the selected epitope on a non-human animal that has been engineered to include parts of a human immune system; and
identifying the selected epitope as a therapeutic to treat the disease in the person.

22. The method of claim 21, wherein obtaining the nucleic acid sequence includes sequencing nucleic acid from the one or more disease-affected cells.

23. The method of claim 22, further comprising sequencing additional nucleic acid from the healthy cells to obtain a healthy-type sequence.

24. The method of claim 23, wherein identifying the plurality of epitopes comprises comparing the sequence to the healthy-type sequence to identify the at least one variant.

25. The method of claim 21, wherein selecting the at least one of the plurality of epitopes based on a predicted MHC binding affinity of that epitope comprises:
providing the plurality of epitopes as an input to a program that predicts affinities using an artificial neural network trained on peptide:MHC affinity measurement data.

26. The method of claim 21, wherein identifying the plurality of epitopes includes translating the nucleic acid sequence into amino acid sequence, excluding portions of the sequence that wholly match the corresponding sequence from the healthy cells, and storing the amino acid sequences in a tangible memory device within the memory system.

27. The method of claim 21, wherein the disease is cancer.

28. The method of claim 21, wherein the non-human animal is an immune-compromised mouse reconstituted with a human immune system.

29. The method of claim 21, wherein the non-human animal is an immune-deficient nude mouse reconstituted with a human immune system.

30. The method of claim 29, wherein the mouse is a Non-Obese Diabetic (NOD) Shi-Scid IL-2R γnull (NOG) mouse.

31. The method of claim 21, wherein the non-human animal is a mouse is reconstituted with human CD34+ cells.

32. The method of claim 31, wherein, after a pre-determined time of reconstitution, the mouse is capable of providing mature human CD45+ cells.

33. The method of claim 32, wherein the mouse comprises hCD3+, hCD4+, and hCD8+ cells.

34. The method of claim 27, wherein the humanized non-human animal is a mouse, wherein the mouse further comprises an xenograft of a human tumor.

35. The method of claim 34, wherein the human tumor xenograft is subcutaneously implanted in the mouse.

36. The method of claim 34, wherein the human tumor xenograft is one selected from the group consisting of: a melanoma tumor graft, a colorectal tumor graft, a breast tumor graft, a lung tumor graft, a xenograft of a human mesenchymal chrondrosarcoma, a xenograft of a human leiomyosarcoma, and a xenograft of a human non-small cell lung cancer.

37. The method of claim 34, wherein the mouse exhibits phenotypic stability of the tumor.

38. The method of any one of claims 1, 18, 19, 20, and 21, further comprising modifying said one or more antigenic epitope peptides to maximize antigenicity.

39. The method of any one of claims 1, 18, 19, 20, and 21, further comprising placing said one or more antigenic epitope peptides in a vector or carrier.
